(19) United States
(12) Patent Application Publication
(10) Pub. No.: US 2013/0289678 A1
(43) Pub. Date: Oct. 31, 2013

(54) THERAPY SYSTEMS INCLUDING HYPERTHERMIC ENERGY DELIVERY ELEMENTS AND CRYOGENIC APPLICATORS AND ASSOCIATED METHODS

(71) Applicants: Benjamin J. Clark, Mountain View, CA (US); Catherine Condie, Shoreview, MN (US); David Francishelli, Anoka, MN (US)

(72) Inventors: Benjamin J. Clark, Mountain View, CA (US); Catherine Condie, Shoreview, MN (US); David Francishelli, Anoka, MN (US)

(73) Assignee: Medtronic Ardian Luxembourg S.a r.1., Luxembourg (LU)

(21) Appl. No.: 13/458,859

(22) Filed: Apr. 27, 2012

(51) Int. Cl. A61F 7/12; A61N 5/00

(52) U.S. Cl. 607/101; 607/105

(57) ABSTRACT

Therapy systems for hyperthermic energy delivery and cryogenic cooling and associated methods of use. A treatment device can have a distal portion with a therapeutic assembly including an energy delivery element and a cryogenic applicator. The energy delivery element can be configured to apply therapeutically-effective hyperthermic energy to a treatment site (e.g., for neuromodulation). The cryogenic applicator can be configured to cool tissue at least proximate the treatment site.

### TECHNICAL FIELD

The present technology relates generally to hyperthermic therapies. In particular, several embodiments are directed to therapy systems including hyperthermic energy delivery elements and cryogenic cooling applicators and associated methods of use.

### BACKGROUND

The sympathetic nervous system (SNS) is a primarily involuntary bodily control system typically associated with stress responses. Fibers of the SNS innervate tissue in almost every organ system of the human body and can affect characteristics such as pupil diameter, gut motility, and urinary output. Such regulation can have adaptive utility in maintaining homeostasis or in preparing the body for rapid response to environmental factors. Chronic activation of the SNS, however, is a common maladaptive response that can drive the progression of many disease states. For example, excessive activation of the renal SNS in particular has been identified experimentally and in humans as a likely contributor to the complex pathophysiology of hypertension, states of volume overload (such as heart failure), and progressive renal disease. For example, radiotracer dilution has demonstrated increased renal norepinephrine (NE) spillover rates in patients with essential hypertension.

Recently, intravascular devices that reduce sympathetic nerve activity by applying an energy field to a target site in the renal artery have been shown to reduce blood pressure in patients with treatment-resistant hypertension. Radiofrequency (RF) energy, for example, can effectively ablate neural fibers that innervate the kidneys. However, RF ablation of the renal nerves can be painful for the patient and typically requires the administration of pain medication or sedation during RF treatment because the targeted sympathetic renal nerves are located proximate efferent pain sensors and fibers. Similar pain-related concerns arise when RF ablation procedures are used for other parts of the body located proximate pain sensors and fibers.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. Furthermore, components may be shown as transparent in certain views for clarity of illustration only and not to indicate that the illustrated component is necessarily transparent.

FIG. 1A is a partially schematic diagram of a therapy system configured in accordance with an embodiment of the present technology.

FIG. 1B illustrates modulating renal nerves with the therapy system of FIG. 1A in accordance with an embodiment of the present technology.

FIG. 2A is a cross-sectional view of a therapeutic assembly in a delivery state within a renal artery in accordance with an embodiment of the present technology.

FIG. 2B is a cross-sectional view of the therapeutic assembly of FIG. 2A in a deployed state within the renal artery in accordance with an embodiment of the present technology.

FIG. 2C is an enlarged cross-sectional view of the therapeutic assembly of FIGS. 2A and 2B in a delivery state within a renal artery in accordance with an embodiment of the present technology.

FIG. 3A is an isometric view of a therapeutic assembly configured in accordance with another embodiment of the present technology.

FIG. 3B illustrates lesions formed at a treatment site by the therapeutic assembly of FIG. 3A.

FIGS. 3C and 3D are schematic diagrams illustrating a treatment site during neuromodulation by the therapeutic assembly of FIG. 3A in accordance with an embodiment of the present technology.

FIG. 4 is a block diagram illustrating a therapeutic method that uses cryogenic cooling to pre-treat a treatment site in accordance with an embodiment of the present technology.

FIG. 5 is a block diagram illustrating a therapeutic method that uses concurrent delivery of hyperthermic energy and cryogenic cooling in accordance with an embodiment of the present technology.

FIG. 6 is a block diagram illustrating a therapeutic method that uses concurrent delivery of hyperthermic energy and cryogenic cooling in accordance with another embodiment of the present technology.

FIG. 7 is a block diagram illustrating a therapeutic method that uses hyperthermic energy to thaw a frozen attachment between a therapeutic device and tissue at a treatment site in accordance with an embodiment of the present technology.

FIG. 8 is a conceptual illustration of the sympathetic nervous system (SNS) and how the brain communicates with the body via the SNS.

FIG. 9 is an enlarged anatomic view of nerves innervating a left kidney to form the renal plexus surrounding the left renal artery.

FIGS. 10A and 10B are anatomic and conceptual views, respectively, of a human body depicting neural efferent and afferent communication between the brain and kidneys.

FIGS. 11A and 11B are anatomic views of the arterial vasculature and venous vasculature, respectively, of a human.

### DETAILED DESCRIPTION

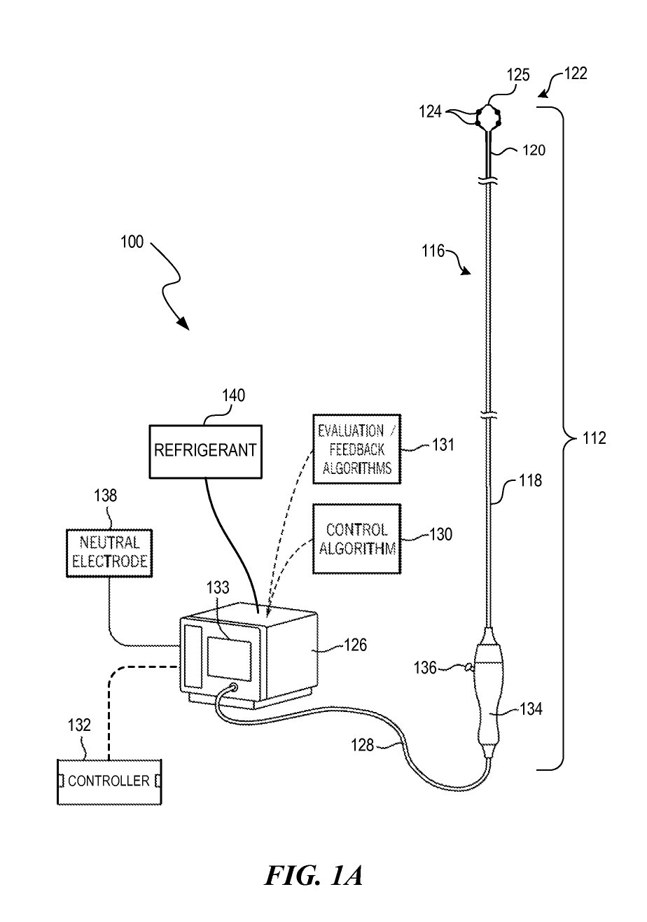

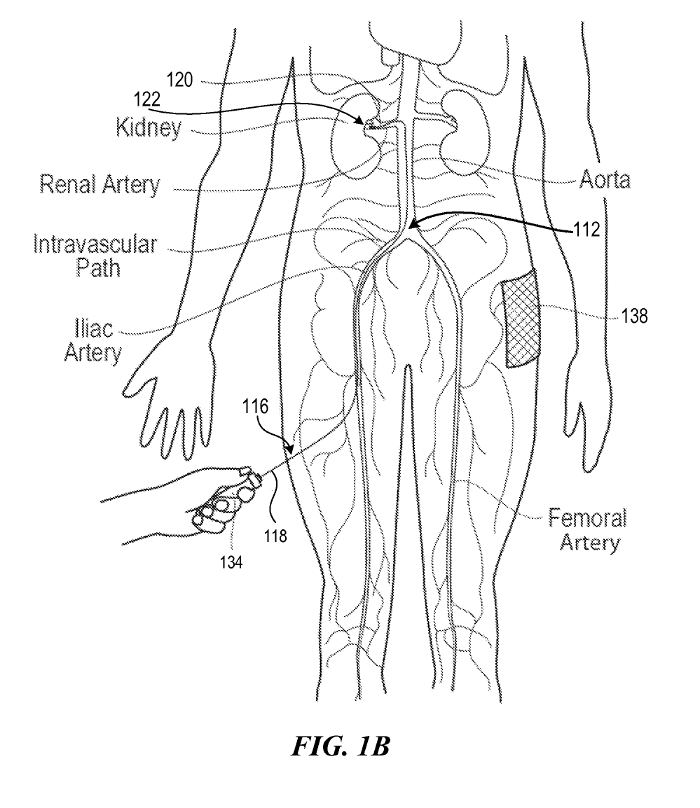

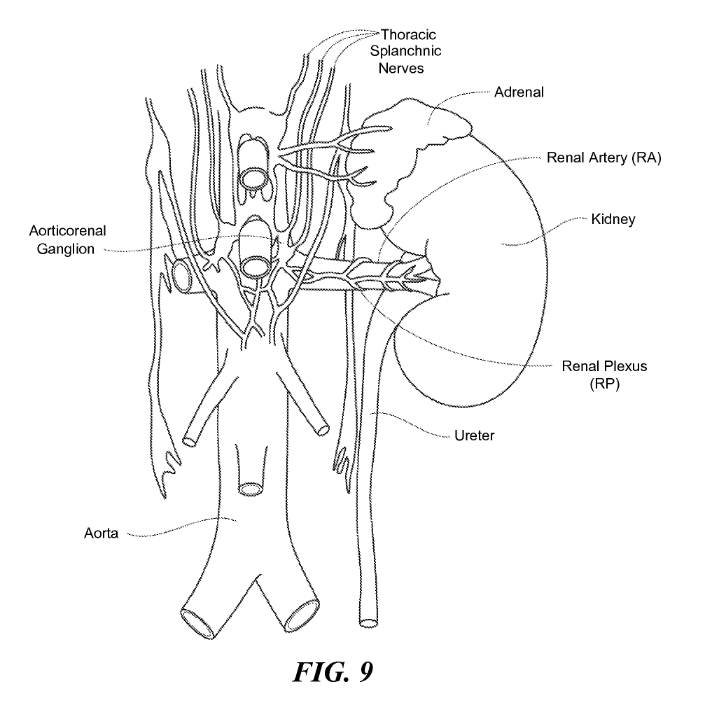

The present technology is generally directed to devices, systems, and methods for therapeutically-effective energy delivery. In various embodiments, treatment devices and systems configured in accordance with the present disclosure can include energy delivery elements for providing therapeutically-effective hyperthermic energy to a treatment site and cryogenic applicators that provide cooling at least proximate the treatment site. As used in the following description, the phrase “at least proximate” can refer to a position near, at, or in a specified location, such as a position near or in a renal artery. Specific details of several embodiments of the technology are described below with reference to FIGS. 1A-11B. Although many of the embodiments are described below with respect to devices, systems, and methods for providing cryogenic cooling and radiofrequency (RF) energy for renal neuromodulation (i.e., rendering neural fibers inert or inactive or otherwise completely or partially reduced in function), other applications (e.g., providing energy for modulating other neural fibers) and other embodiments (e.g., using other forms of energy and methods for inducing hypothermic and hyperthermic responses at various treatment sites, such as the heart, liver, bladder, prostate, ovaries and other blood vessels) in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below with reference to FIGS. 1A-11B.

The terms “distal” and “proximal” are used in the following description with respect to a position or direction relative to the operator or the operator's control device (e.g., a handle assembly). “Distal” or “distally” are a position distant from or in a direction away from the operator or the operator's control device. “Proximal” and “proximally” are a position near or in a direction toward the operator or the operator's control device.

### I. NEUROMODULATION

Neuromodulation is the partial or complete incapacitation or other effective disruption of nerves. In particular, neuromodulation comprises inhibiting, reducing, and/or blocking neural communication along neural fibers (i.e., efferent and/or afferent nerve fibers). Such incapacitation can be long-term (e.g., permanent or for periods of months, years, or decades) or short-term (e.g., for periods of minutes, hours, days, or weeks). For example, renal neuromodulation refers to rendering the nerves that innervate the kidney inert, inactive, or otherwise completely or partially reduced in function to treat hypertension. Neuromodulation may also be used at other target sites within the human body to treat various other pathologies. For example, neuromodulation (e.g., denervation) can target the splenic artery for treatment of auto-immune diseases, the anterior choroidal artery for treatment of Parkinson's disease, the celiac ganglia/plexus for treatment of pancreatic pain, the stellate ganglia for treatment of ventricular arrhythmia or Raynaud's disease, the ovarian artery for treatment of infertility and polycystic ovary syndrome, the uterine artery or cystic artery for treatment of over-active bladder, the mesenteric arteries for treatment of Chrohn's disease, ulcerative colitis, or gastrointestinal dysmotility, and/or other target sites within the human body.

Various techniques can be used to partially or completely incapacitate neural pathways, such as those innervating the kidney. The purposeful application of energy (e.g., electrical and/or thermal energy) to tissue by an energy delivery element can induce one or more desired thermal heating effects on localized regions of tissue (e.g., the renal artery and adjacent regions of the renal plexus, which lay intimately within or adjacent to the adventitia of the renal artery). The purposeful application of the thermal heating effects can achieve neuromodulation.

The thermal heating effects can include both thermal ablation and non-ablative thermal alteration or damage (e.g., via sustained heating and/or resistive heating). Desired thermal heating effects may include raising the temperature of target neural fibers above a desired threshold to achieve non-ablative thermal alteration, or above a higher temperature to achieve ablative thermal alteration. For example, the target temperature can be above body temperature (e.g., approximately 37° C.) but less than about 45° C. for non-ablative thermal alteration, or the target temperature can be about 45° C. or higher for the ablative thermal alteration.

More specifically, exposure to thermal energy (heat) in excess of a body temperature of about 37° C., but below a temperature of about 45° C., may induce thermal alteration via moderate heating of the target neural fibers or of vascular structures that perfuse the target fibers. In cases where vascular structures are affected, the target neural fibers are denied perfusion resulting in necrosis of the neural tissue. For example, this may induce non-ablative thermal alteration in the fibers or structures. Exposure to heat above a temperature of about 45° C., or above about 60° C., may induce thermal alteration via substantial heating of the fibers or structures. For example, such higher temperatures may thermally ablate the target neural fibers or the vascular structures. In some patients, it may be desirable to achieve temperatures that thermally ablate the target neural fibers or the vascular structures, but that are less than about 90° C., or less than about 85° C., or less than about 80° C., and/or less than about 75° C. Regardless of the type of heat exposure utilized to induce the thermal neuromodulation, a reduction in sympathetic nerve activity (e.g., renal sympathetic nerve activity (RSNA)) is expected.

Hypothermic effects may also provide neuromodulation. Cryotherapy, for example, may be used to cool tissue at a target site to provide therapeutically-effective direct cell injury (e.g., necrosis), vascular injury (e.g., starving the cell from nutrients by damaging supplying blood vessels), and sublethal hypothermia with subsequent apoptosis. Exposure to cryotherapeutic cooling can cause acute cell death (e.g., immediately after exposure) and/or delayed cell death (e.g., during tissue thawing and subsequent hyperperfusion). Embodiments of the present technology can include cooling a structure at or near an inner surface of a renal artery wall such that proximate (e.g., adjacent) tissue is effectively cooled to a depth where sympathetic renal nerves reside. For example, the cooling structure is cooled to the extent that it causes therapeutically effective, cryogenic renal-nerve modulation. Sufficiently cooling at least a portion of a sympathetic renal nerve is expected to slow or potentially block conduction of neural signals to produce a prolonged or permanent reduction in renal sympathetic activity.

Cryotherapy has certain characteristics that can be beneficial for intravascular renal neuromodulation. For example, cryotherapies generally operate at temperatures that cause cryotherapeutic applicators to adhere to moist tissue. This can be beneficial because it promotes stable, consistent, and continued contact during treatment. The typical conditions of treatment can make this an attractive feature because, for example, a patient can move during treatment, a catheter associated with an applicator can move, and/or respiration can cause the kidneys to rise and fall and thereby move the renal arteries. In addition, blood flow is pulsatile and causes the renal arteries to pulse. Adhesion associated with cryotherapeutic cooling also can be advantageous when treating short renal arteries in which stable intravascular positioning can be more difficult to achieve.

### II. SELECTED EMBODIMENTS OF THERAPY SYSTEMS

FIG. 1A illustrates a therapy system 100 (“system 100”) configured in accordance with an embodiment of the present technology. The system 100 can include a treatment device 112 operably coupled to an energy source or console 126 via a line or cable 128. The console 126 can be integrated into a single unit as shown in FIG. 1A, or the console 126 may include separate and distinct components operably coupled to one another and/or to the treatment device 112. In the embodiment shown in FIG. 1A, the treatment device 112 includes an elongated catheter shaft 116 having a proximal portion 118, a handle assembly 134 at a proximal region of the proximal portion 118, and a distal portion 120 extending distally relative to the proximal portion 118. The treatment device 112 further includes a treatment section or therapeutic assembly 122 at or near the distal portion 120 of the catheter shaft 116. As described in further detail below, the therapeutic assembly 122 can be configured to provide therapeutically-effective energy for neuromodulation (e.g., ablation or non-ablation neuromodulation) using both hyperthermic and hypothermic applicators. The therapeutic assembly 122, for example, can include one or more energy delivery elements 124 (e.g., electrodes) that deliver therapeutically-effective hyperthermic energy (e.g., RF energy) to a treatment site and a cryogenic applicator 125 that cools tissue and/or other structures (e.g., the energy delivery elements 124) at least proximate the treatment site. The energy delivery elements 124 may also be used to stimulate and/or sense neural activity at the treatment site before, during, and/or after neuromodulation. In other embodiments, the therapeutic assembly 122 may include other sensors to detect neural activity, temperature, and/or other properties proximate the treatment site.

In certain embodiments, the therapeutic assembly 122 can be delivered intravascularly to a treatment site in a delivery state or arrangement (e.g., a low-profile or collapsed configuration). For example, the distal end of the therapeutic assembly 122 may define a passageway for engaging a guide wire (not shown) to deliver the distal portion 120 and/or the therapeutic assembly 122 to the treatment site using over-the-wire (“OTW”) or rapid exchange (“RX”) techniques. At the treatment site, the therapeutic assembly 122 can transform to a deployed state or arrangement (e.g., an expanded configuration) in which the therapeutic assembly 122 can deliver hyperthermic energy and/or cooling to the treatment site to provide therapeutically-effective electrically- and/or thermally-induced neuromodulation. In various embodiments, the therapeutic assembly 122 may be placed or transformed between the delivery and deployed states via remote actuation using an actuator 136 (e.g., a knob, pin, or lever) carried by the handle assembly 134 and/or using other suitable mechanisms or techniques. For example, the therapeutic assembly 122 may be delivered to the treatment site within a guide sheath (not shown), which may be at least partially retracted or otherwise removed from the therapeutic assembly 122 to allow it to move from the low-profile delivery state to the expanded deployed state.

The console 126 can be configured to generate selected forms and magnitudes of energy for delivery to the treatment site via the therapeutic assembly 122. For example, the console 126 can include an RF energy generator that is electrically coupled to one or more of the energy delivery elements 124 via supply wires (not shown) that pass along the catheter shaft 116 or through a lumen in the catheter shaft 116 to transmit RF energy to the energy delivery elements 124. The RF energy can be delivered in a monopolar electric field or a bipolar electric field via the energy delivery element 124. In monopolar embodiments, a neutral or dispersive electrode 138 may be electrically connected to the console 126 and attached to the exterior of the patient (e.g., as shown in FIG. 1B). In other embodiments, the console 126 can provide microwave energy, ultrasound energy, high intensity focused ultrasound (HIFU) energy, direct current (DC), laser, electroporation, and/or other suitable sources of hyperthermic energy to the energy delivery elements 124.

As shown in FIG. 1A, the console 126 can further include and/or be in fluid connection with a supply of refrigerant 140, such as nitrous oxide (N₂O), Argon (Ar), liquid nitrogen (N₂), carbon dioxide (CO₂), chlorofluorocarbon, hydrochlorofluorocarbon, other hydrofluorocarbons, and/or other suitable refrigerants for cryogenic cooling. In other embodiments, for example, the supply of refrigerant 140 can include a refrigerant known as R-410A, which is a near-azeotropic mixture of difluoromethane (CH₂F₂; known as R-32) and pentafluoroethane (CHF₂CF₃; known R-125). The refrigerant 140 can be stored in a cartridge (e.g., a single-use cartridge) or a canister (e.g., a tank, cylinder, or other suitable containers that are not cartridges), and can be housed in the console 126 or fluidly coupled to the console 126. In other embodiments, the refrigerant 140 can be fluidly coupled directly to a portion of the treatment device 112 (e.g., stored in or attached to the handle 134).

The refrigerant 140 can be in fluid communication with the cryogenic applicator 125 of the therapeutic assembly 122 to provide cooling at a zone at least proximate the treatment site. For example, the cryogenic applicator 125 can provide cooling to surrounding tissue and/or the energy delivery elements 124. The refrigerant 140 can be delivered to the cryogenic applicator 125 via a supply line (not shown; e.g., extending along or through the catheter shaft 116) and, optionally, a supply control valve can be operably coupled to the supply line to manually or automatically control the flow of the refrigerant 140 along the supply line. An exhaust line (not shown) can be placed in fluid communication with the cryogenic applicator 125 (e.g., running parallel to or incorporated with the line 128) and configured to receive exhausted refrigerant from the cryogenic applicator 125. In various embodiments, the exhaust line can be operably coupled to a pump (e.g., a vacuum pump, a DC-powered pump, etc.), a back-pressure control valve, and/or other suitable features for controlling cryogenic cooling therapies. The system 100 can include additional or other features associated with cryogenic cooling therapies, such as those described in U.S. patent application Ser. No. 13/279,330, filed Oct. 23, 2011, and entitled “NEUROMODULATION CRYOTHERAPEUTIC DEVICES AND ASSOCIATED SYSTEMS AND METHODS,” which is herein incorporated by reference in its entirety.

As further shown in FIG. 1A, the console 126 can include and/or be operably connected to a controller 132 (e.g., a handheld controller, key pad, foot pedal, etc.) to allow the operator to initiate, terminate and, optionally, adjust various operational characteristics of the console 126 (e.g., power delivery). For example, the controller 132 can be configured to pre-treat tissue at the treatment site with cryogenic cooling (e.g., to reduce the impedance of the tissue, therapeutically modulate pain neurons, etc.), and subsequently deliver therapeutically-effective hyperthermic energy to modulate renal nerves. The console 126 can also be configured to deliver therapeutic procedures via an automated control algorithm 130 and/or under the control of a clinician. For example, the console 126 can include one or more computing devices (e.g., personal computers, server computers, tablets, etc.) having processing circuitry (e.g., a microprocessor) that are configured to execute stored instructions relating to the control algorithm 130. In addition, the processing circuitry may be configured to execute one or more evaluation/feedback algorithms 131 and may provide feedback to the user. A display 133 and/or associated features may be configured to provide indications of power levels, temperature readings, visual and/or audio indications, and/or may be configured to communicate the information to another device, such as a monitor in a catheterization laboratory.

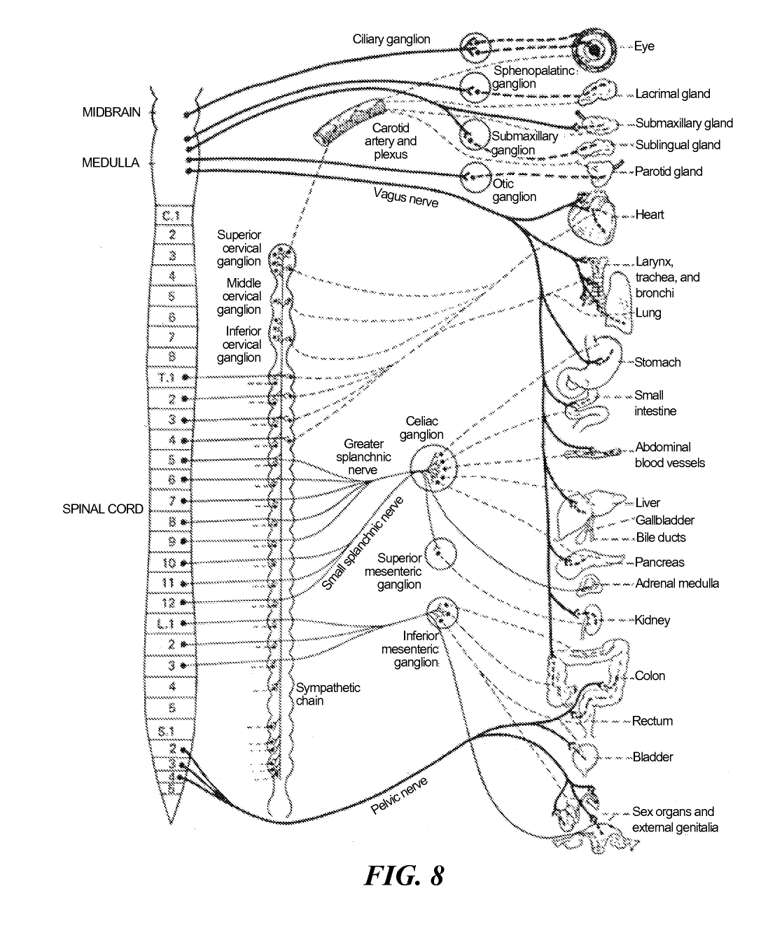

The system 100 may be used for neuromodulation at various target sites throughout the body. For example, FIG. 1B (with additional reference to FIG. 8) illustrates modulating renal nerves with an embodiment of the system 100 of FIG. 1A. The treatment device 112 can provide access to the renal plexus RP through an intravascular path, such as from a percutaneous access site in a femoral (illustrated), brachial, radial, axillary or other artery to a targeted treatment site within a respective renal artery RA. As illustrated, a section of the proximal portion 118 of the catheter shaft 116 is exposed externally of the patient. By manipulating the proximal portion 118 of the catheter shaft 116 from outside the intravascular path (e.g., via the handle assembly 134), the operator may advance the catheter shaft 116 through the sometimes tortuous intravascular path and remotely manipulate or actuate the distal portion 120 of the catheter shaft 116. Image guidance, e.g., computed tomography (CT), fluoroscopy, intravascular ultrasound (IVUS), optical coherence tomography (OCT), or another suitable guidance modality, or combinations thereof, may be used to aid the operator's manipulation. Further, in some embodiments, image guidance components (e.g., IVUS, OCT) may be incorporated into the treatment device 112 itself.

After the therapeutic assembly 122 is adequately positioned in the renal artery RA, it can be deployed (e.g., radially expanded) and manipulated using the handle 134 or other suitable means until the therapeutic assembly 122 is positioned at its target site in stable contact with the inner wall of the renal artery RA, as will be described in further detail below. The purposeful application of by the energy by the energy delivery element(s) 124 (FIG. 1A) to tissue of the renal artery RA in conjunction with cooling provided by the cryogenic applicator 125 (FIG. 1A) is expected to induce one or more desired neuromodulating effects on localized regions of the renal artery RA and adjacent regions of the renal plexus RP, which lay intimately within, adjacent to, or in close proximity to the adventitia of the renal artery RA. The purposeful application of the hyperthermic energy and cryogenic cooling may achieve neuromodulation along all or at least a portion of the renal plexus RP. In other embodiments, the treatment device 112 may be intravenously introduced such that the therapeutic assembly 122 can apply hyperthermic energy and cryogenic cooling to dennervate or otherwise modulate nerves proximate a renal vein. In further embodiments, the therapeutic assembly 122 may be used for neuromodulation elsewhere in the body. For example, the therapeutic assembly 122 may be used in various cardiac applications to destroy abnormal electrical pathways for atrial fibrillation (AF), supraventricular tachycardia (SVT), atrial tachycardia, etc.). The therapeutic assembly 122 may also be used to treat tumors in the prostate, liver, lungs, and/or other biological structures.

The neuromodulating effects are generally a function of, at least in part, power, time, temperature, contact between the therapeutic assembly 122 (e.g., the energy delivery elements 124) and the vessel wall, and blood flow through the vessel. The neuromodulating effects may include denervation, thermal ablation, and non-ablative thermal alteration or damage (e.g., via sustained heating and/or resistive heating). Desired thermal heating effects may include raising the temperature of target neural fibers above a desired threshold to achieve non-ablative thermal alteration, or above a higher temperature to achieve ablative thermal alteration. For example, the target temperature may be above body temperature (e.g., approximately 37° C.) but less than about 45° C. for non-ablative thermal alteration, or the target temperature may be about 45° C. or higher for the ablative thermal alteration. Desired non-thermal neuromodulation effects may include altering the electrical signals transmitted in a nerve.

### III. THERAPEUTIC ASSEMBLIES FOR HYPERTHERMIC ENERGY DELIVERY AND CRYOGENIC COOLING

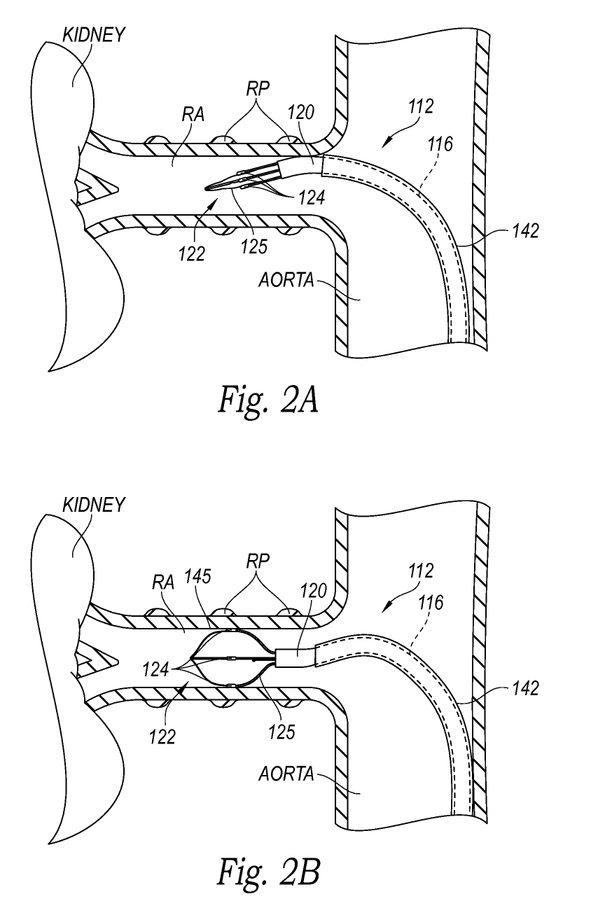

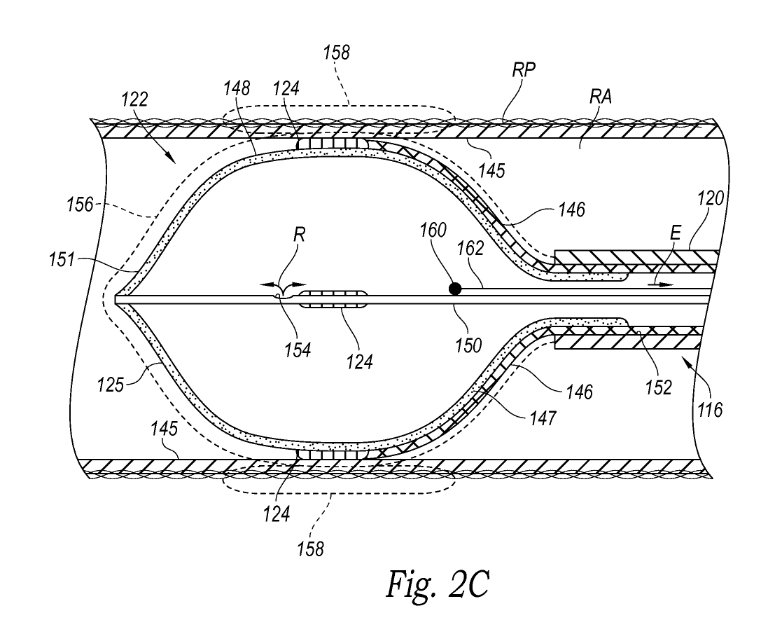

FIG. 2A is an enlarged cross-sectional view of the therapeutic assembly 122 of FIGS. 1A and 1B in a delivery state (e.g., a low-profile or collapsed configuration) within a renal artery RA in accordance with an embodiment of the present technology. The delivery state of the therapeutic assembly 122 can facilitate delivery (e.g., insertion) and/or removal of the therapeutic assembly 122 and, in certain embodiments, repositioning of the therapeutic assembly 122 at the treatment site. For example, in the embodiment illustrated in FIG. 2A, the therapeutic assembly 122 has a delivery state sized and shaped to navigate into and out of the renal artery RA. In other embodiments, the therapeutic assembly 122 can have a low-profile or other delivery configuration shaped and sized to fit within other portions of the vasculature and/or other structures within the body.

As shown in FIG. 2A, at least a portion of the treatment device 112 can be placed in a guide sheath 142 that flexes and/or otherwise facilitates navigation through the vasculature to locate the distal portion 120 of the catheter shaft 116 proximate a treatment site, e.g., within the renal artery RA. For example, a 6 French pre-formed guide catheter with a lumen diameter of approximately 1.80 mm (0.071 inch) may be used as guide sheath 142. A medical guide wire (not shown) can be used in addition to or in lieu of the guide sheath 142 to facilitate delivery of the treatment device 112 to the treatment site. For example, a guide wire can be inserted through the vasculature to the treatment site, and the catheter shaft 116 and/or the guide sheath 142 can be passed over the guide wire to the treatment site. At the treatment site, the guide sheath 142 and the catheter shaft 116 can be moved relative to one another to expose the therapeutic assembly 122. For example, the guide sheath 142 can be pulled proximally or otherwise retracted from the distal end of the catheter shaft 116, and/or the catheter shaft 116 can be pushed distally from the opening of the guide sheath 142. In various embodiments, the therapeutic assembly 122 may deploy automatically (e.g., using a shape memory material) as it is exposed from the guide sheath 142. In other embodiments, the therapeutic assembly 122 can remain in a substantially low-profile configuration until the operator initiates deployment (e.g., via the actuator 136 shown in FIGS. 1A and 1B).

FIG. 2B is an enlarged cross-sectional view of the therapeutic assembly 122 of FIG. 2A in a deployed state (e.g., an expanded configuration) within the renal artery RA in accordance with an embodiment of the present technology. The therapeutic assembly 122 can expand radially outward in the deployed state such that the energy delivery elements 124 at the exterior of the cryogenic applicator 125 press against or otherwise contact the inner surface of a vessel wall 145 of the renal artery RA. For example, as described in further detail below, the cryogenic applicator 125 can define at least a portion of an expansion chamber in which a refrigerant (e.g., refrigerant 140 described with reference to FIG. 1A) expands or otherwise flows to provide cryogenic cooling. As shown in FIG. 2B, in certain embodiments, the cryogenic applicator 125 can expand such that the therapeutic assembly 122 at least partially occludes blood flow proximate the treatment site (e.g., in the renal artery RA). This configuration can reduce the variability of the neuromodulation procedures associated with blood flow, such as unstable electrical contact with the arterial wall 145, changes in temperature, etc.

FIG. 2C is an enlarged cross-sectional view illustrating further features of the therapeutic assembly 122 of FIGS. 2A and 2B in the deployed state in accordance with an embodiment of the present technology. For example, as shown in FIG. 2C, the therapeutic assembly 122 can include a plurality of conductive leads 146 (e.g., electrical wires) that extend through or along the catheter shaft 116 to corresponding energy delivery elements 124. The leads 146 can operably couple the energy delivery elements 124 to an energy source (e.g., an RF energy generator) at the proximal portion 118 of the catheter shaft 116 (FIG. 1A). As shown in FIG. 2C, the energy delivery elements 124 can be discrete electrodes that are spaced apart from one another on an outer surface 148 of the cryogenic applicator 125 to provide either non-circumferential ablation or circumferential ablation depending upon the size of each energy delivery element's ablation zone. In FIG. 2C, for example, three energy delivery elements 124 are shown, but the therapeutic assembly 122 can include fewer than three energy delivery elements 124 (e.g., one or two energy delivery elements 124) or more than three energy delivery elements 124. In further embodiments, the energy delivery elements 124 may include one or more loop electrodes (not shown) that extend partially and/or completely around a circumference the outer surface 148 of the cryogenic applicator 125. In one embodiment, for example, two loop electrodes can be spaced laterally apart from one another on the outer surface 148 and provide a bipolar RF energy field and/or other suitable forms of electric energy. In other embodiments, the energy delivery elements 124 can have other suitable shapes or configurations, such as a helical shape (not shown) around the cryogenic applicator 125 with a plurality of energy delivery elements 124 positioned thereon, flexible (e.g., bendable) energy delivery elements 124, and/or longitudinally offset energy delivery elements 124. In further embodiments, the one or more energy delivery elements 124 can include a HIFU transducer at the distal portion 120 of the catheter shaft 116, such as the catheter-based transducer disclosed in U.S. Pat. No. 6,669,655, filed Oct. 19, 2000, and entitled “SONIC ELEMENT AND CATHETER INCORPORATING SAME,” which is herein incorporated by reference in its entirety. In this embodiment, a HIFU energy delivery element (not shown) may have a cylindrical shape and may be configured to be positioned a distance apart from the tissue of the vessel wall 145. In other embodiments, the HIFU energy delivery element 126 may have other suitable shapes and/or be configured to contact the vessel wall 145.

The cryogenic applicator 125 can include an expandable member 147 (e.g., a balloon) that defines an expansion chamber for cryogenic cooling and is movable from the delivery state (FIG. 2A) to the deployed state (FIGS. 2B and 2C). The expandable member 147 can comprise a compliant material (e.g., polyurethane), a non-compliant material (e.g., nylon), and/or a combination of compliant and non-compliant materials known to those of skill in the art of dilative or occlusive balloon catheters. In various embodiments, for example, the expandable member 147 can be made from polyurethane and/or other compliant or semi-compliant materials that can expand and conform to vessel walls to fully occlude vessels of varying sizes (e.g., vessels having an inner diameter from approximately 3 mm to approximately 10 mm, or in specific applications approximately 4 mm to approximately 8 mm). In other embodiments, the expandable member 147 can be made from nylon and/or other non-compliant materials and sized to accommodate vessels within a certain size range. For example, a non-compliant nylon expandable member 147 can be sized to accommodate vessels having an inner diameter between approximately 3 mm and 6 mm, and a larger non-compliant nylon expandable member can be sized to accommodate vessels having an inner diameter between approximately 7 mm and 10 mm.

In the embodiment illustrated in FIG. 2C, the cryogenic applicator 125 is configured for use in the renal artery RA. Accordingly, the cryogenic applicator 125 can be relatively short in the deployed state (e.g., 10 mm or less) to accommodate the length and tortuosity of the renal artery RA (e.g., typically between 4-6 cm) and can have a diameter in an expanded configuration large enough to contact at least a portion of the inner circumference of the arterial wall 145 (e.g., between 3-10 mm in diameter). In other embodiments, the cryogenic applicator 125 can have other suitable dimensions configured for other treatment sites in the vasculature (e.g., in the hepatic artery, splenic artery, mesenteric artery, or anterior choroidal artery, etc.) and/or elsewhere in the body.

The cryogenic applicator 125 or a portion thereof (e.g., the expandable member 147) can be in fluid communication with a supply tube or lumen 150 and an exhaust tube or lumen 152 that extend along at least a portion of the catheter shaft 116. In the embodiment illustrated in FIG. 2C, the supply lumen 150 extends beyond the distal portion 120 of the catheter shaft 116 and connects to a distal end portion 151 of the cryogenic applicator 125 (e.g., a distal portion of the expandable member 147) to provide stability and/or limit axial expansion of the expandable member 147. In other embodiments (not shown), the supply lumen 150 may terminate proximate to or within the cryogenic applicator 125, and the distal end portion 151 may be supported by another suitable structure (e.g., an inner core wire or a shaft having a guide wire lumen) or remain unsupported to provide a continuous, gentle surface with which to contact vessel walls.

The supply lumen 150 can be fluidly coupled to a refrigerant source (e.g., a refrigerant cartridge or canister) at its proximal end portion (not shown) and may be sized to retain at least a portion of the refrigerant that reaches the expansion chamber at a high pressure liquid state. The supply lumen 150 can include an orifice 154 from which refrigerant can expand into the expansion chamber (e.g., as indicated by the arrows R), or refrigerant can be configured to expand from a distal opening of a capillary tube (not shown) extending from the supply lumen 150. In various embodiments, the orifice 154 may have a cross-sectional area less than that of the supply lumen 150 to impede the flow of refrigerant proximate the expansion chamber, thereby increasing the pressure drop of the refrigerant entering the expansion chamber and concentrating the refrigeration power at the cryogenic applicator 125. For example, the orifice 154 can be sized relative to the area and/or length of the exhaust lumen 152 at the distal portion 120 of the catheter shaft 116 to provide a sufficient flow rate of refrigerant, to produce a sufficient pressure drop when the refrigerant enters the expansion chamber, and to allow for sufficient venting of expanded refrigerant (e.g., indicated by the arrow E) through the exhaust lumen 152 to establish and maintain cooling at the cryogenic applicator 125. In other embodiments, the supply lumen 150 can include a plurality of orifices 154 (shown in broken lines) spaced apart from each other axially along and/or circumferentially around the supply lumen 150.

In operation, a liquid refrigerant R can expand into a gaseous phase as it passes through the orifice 154 of the supply lumen 150 into the expansion chamber (defined within at least a portion of the expandable member 147), thereby inflating the expandable member 147 and the therapeutic assembly 122 therewith. The expansion of the refrigerant causes a temperature drop in the expansion chamber, thereby forming a cooling zone 156 (shown in broken lines) around at least a portion of the cryogenic applicator 125. The cryogenic applicator 125 can be configured to form the cooling zone 156 before, during, and/or after the delivery of hyperthermic energy by the energy delivery elements 124. For example, concurrently with the application of hyperthermic energy via the energy delivery elements 124, the cooling zone 156 can be provided at a relatively low refrigeration power, e.g., a power less than that required to induce neuromodulation. The cooling zone 156 can cool the energy delivery elements 124 and/or the body tissue at or proximate the treatment site (e.g., the inner surface of arterial wall 145). In other embodiments, all or a portion of the refrigerant can exit the orifice 154 of the supply lumen 150 in a liquid state and evaporate at or near the outer limits of the cooling zone 156 to increase the refrigeration power near the area of contact to the arterial wall. As described in further detail below, in further embodiments the cooling zone 156 can be provided by a non-expandable cryotherapeutic applicator, such a cryoprobe at the distal portion 120 of the catheter shaft 116 (e.g., a FREEZOR catheter available from Medtronic, Inc. of Minneapolis, Minn.), the energy delivery elements 124 may optionally also be positioned at the distal portion 120 of the catheter shaft 116 (e.g., in a spiral and/or other suitable arrangement).

The resistive heating in and/or at the tissue provided by the energy delivery elements 124 can raise the temperatures at hyperthermic zones 158 (shown in broken lines) in the wall 145 of the renal artery RA and the neural fibers of the surrounding renal plexus RP to provide therapeutically-effective neuromodulation. As shown in FIG. 2C, the cooling zone 156 provided by the cryogenic applicator 125 is expected to maintain lower temperatures, and thereby reduce thermal trauma in the tissue proximate the inner surface of the vessel wall 145 during hyperthermic neuromodulation. On the other hand, the hyperthermic zone 158 can extend or focus more on the exterior area of the vessel wall 145 where the nerves reside. Therefore, the therapeutic assembly 122 can provide a reverse thermal gradient across a portion of the vessel wall 145 to provide hyperthermic neuromodulation at a depth in the tissue, while reducing potential hyperthermal effects on the vessel tissue closer to the therapeutic assembly 122.

As shown in FIG. 2C, the expanded cryogenic applicator 125 may be sized to occlude a vessel (e.g., the renal artery RA) proximate the treatment site while pressing the energy delivery elements 124 in apposition with the adjacent vessel wall 145. In selected embodiments, the cryogenic applicator 125 may be configured to further stabilize and secure the contact between the energy delivery elements 124 and the tissue at the target site by establishing a temporary frozen attachment between at least a portion of the therapeutic assembly 122 and vessel tissue adjacent the target site. For example, the cryotherapeutic applicator 125 can maintain a temperature below the freezing temperature of water (0° C.) along a portion of its outer surface 148 that contacts the vessel wall 145, and/or other portions of the therapeutic assembly 122 (e.g., a deactivated energy delivery element 124) such that the therapeutic assembly 122 freezes to the surrounding tissue. The stability of the contact between the energy delivery elements 124 and the adjacent vessel wall 145 via freezing and/or contact pressure applied by the expandable member 147, along with the reduced blood flow variability provided by occluding the vessel, enhances the consistency with which the therapeutic assembly 122 can deliver hyperthermic energy for neuromodulation.

In various embodiments, the cryogenic applicator 125 can be configured to cool one or more of the energy delivery elements 124 during hyperthermic energy delivery. A cooled energy delivery element 124 can apply power for a longer period of time and/or a higher power at the treatment site because the cooling reduces or eliminates the hyperthermic effects to the adjacent tissue, and the extended treatment time can form deeper hyperthermic lesions in neural tissue or other tissue at the treatment site. However, too much cooling can increase the impedance of the targeted tissue (e.g., by forming ice in the tissue), and thereby inhibit the intended lesion formation. Accordingly, in embodiments with concurrent cryogenic cooling and hyperthermic energy delivery, the temperature of the cryogenic applicator 125 proximate the cooled energy delivery elements 124 (e.g., inward of and around the energy delivery elements 124) can be maintained above the freezing temperature of water, such as about 15-35° C. and/or other suitable temperature ranges to maintain a relatively low impedance. In such embodiments, the therapeutic assembly 122 may further include one or more temperature sensors 160 (e.g., a thermocouple, thermistor, etc.) in and/or on the expandable member 147 and operably coupled to the controller 132 (FIG. 1A) via a supply wire or lead 162 that extends through the catheter shaft 116 to transmit signals from the sensor(s) 160. The controller 132 can be configured to automatically or manually manipulate the refrigerant flow into the cryogenic applicator 125 and/or associated cooling components to maintain the designated temperature as monitored via the sensor 160. In other embodiments, the therapeutic assembly 122 can include other or additional sensors, such as pressure sensors, impedance sensors, optical sensors, flow sensors, and/or other suitable sensors (not shown) to facilitate monitoring and controlling the therapeutic treatment.

Several embodiments of the therapeutic assembly 122 can pre-treat tissue at the treatment site via the cryogenic applicator 125 before delivering hyperthermic energy. For example, the cryogenic applicator 125 can cool and/or freeze tissue at or near the treatment site before the application of hyperthermic energy to at least temporarily block or desensitize adjacent pain neurons and/or otherwise inhibit the transmission of action potentials to the central nervous system (CNS). This temporary effect on the pain neurons can significantly reduce the level of pain experienced by the patient during the subsequent hyperthermic energy delivery. Therefore, the therapeutic assembly 122 can be well-suited for treatment sites in which pain sensors and associated nerve fibers are located nearby. For example, the therapeutic assembly 122 can be used during renal neuromodulation to pre-treat tissue at the renal artery RA because efferent, pain sensors are located very near the targeted sympathetic nerves.

Pre-treating the treatment site with the cryogenic applicator 125 can also decrease the electrical impedance of the targeted tissue, and therefore improve the subsequent transmission of electrical energy via the energy delivery elements 124. Without being bound by theory it is thought that cell membranes largely contribute to the impedance of at least lower-frequency electrical signals and that breaking down cell walls (e.g., using freeze-thaw cycles until the tissue becomes edematous) can reduce the electrical impedance and increase the conductivity of the tissue. Accordingly, in several embodiments of the present technology, the cryogenic applicator 125 can be used to cryogenically cool tissue at the target site before the application of hyperthermic energy to break down and/or otherwise alter cell walls. The duration and temperature of the cooling can be selected based on the tissue properties at the treatment site, the intended power of the subsequently applied RF energy, and/or other operational parameters. In some embodiments, the cryogenic applicator 125 can cool tissue at the treatment site for about 1-5 minutes at about −55° C., which can decrease the magnitude of both imaginary and real components of the complex electrical impedance of the tissue. For example, laboratory experiments have shown that cryogenically pre-treating tissue at about −55° C. for 1-5 minutes can reduce the magnitude of the imaginary component of tissue impedance by about 40% to over 80% and can reduce the magnitude of the real component of tissue impedance by about 10% to over 70% depending upon the duration of the cryogenic cooling and the power of the subsequently applied hyperthermic energy. In other embodiments, the cooling time may be longer or shorter and/or the cooling temperature may be higher or lower based, at least in part, on operational parameters (e.g., power, lesion depth, time, etc.). In further embodiments, the cryogenic applicator 125 can be configured to apply a series of cooling and thawing cycles to the treatment site (e.g., 2 minutes cool, 2 minutes thaw, 2 minutes cool) to provide the desired change in electrical impedance.

A decrease in the electrical impedance of the targeted tissue, such as the effect provided by using the cryogenic applicator 125, can provide better transmission of the subsequently-applied hyperthermic energy, and thereby allow the energy delivery elements 124 to modulate neural fibers to a greater extent and/or at greater depths in the tissue (i.e., a distance from the energy delivery elements 124) than without the cryogenic applicator 125. For example, laboratory experiments using ex vivo bovine ventricular tissue have shown that cryogenically pre-treating tissue at approximately −60° C. for a period of about 5 minutes can increase the lesion depth of a bipolar RF energy delivery element 15-65%. The reduced electrical impedance of the tissue can also increase the speed of the hyperthermic energy delivery, and thereby increase the efficiency of the therapeutic assembly 122 overall.

In addition, it is thought that the largest reduction in tissue impedance caused by cryogenic cooling primarily comes from the reactance or capacitive component, but that the magnitude of the impedance and the real component of the tissue impedance are also decreased. The combination of the reactive and real components of impedance may be used with imaging systems (e.g., with the display 133 of FIG. 1A) to differentiate between cryo-induced lesions, hyperthermic lesions, and/or healthy tissue. Moreover, the reduced electrical impedance of the pre-treated tissue (e.g., frozen or thawed tissue) in conjunction with the subsequent phase change of the tissue components (e.g., cytoplasm and extracellular liquid) from a solid to a liquid during hyperthermic lesion formation can enhance the correlation between electrical impedance and hyperthermic lesion creation and size. Accordingly, the impedance of the treatment site can be measured during hyperthermic energy delivery to provide real-time monitoring of the hyperthermic lesion. Furthermore, the changes in tissue impedance caused by cryogenic cooling can also be used to map one or more treatment sites to determine their viability at treatment sites (e.g., whether the tissue has a suitable low impedance) before initiating hyperthermic energy delivery. This allows the clinician to select a treatment site with desired characteristics without damaging tissue at a non-viable treatment site.

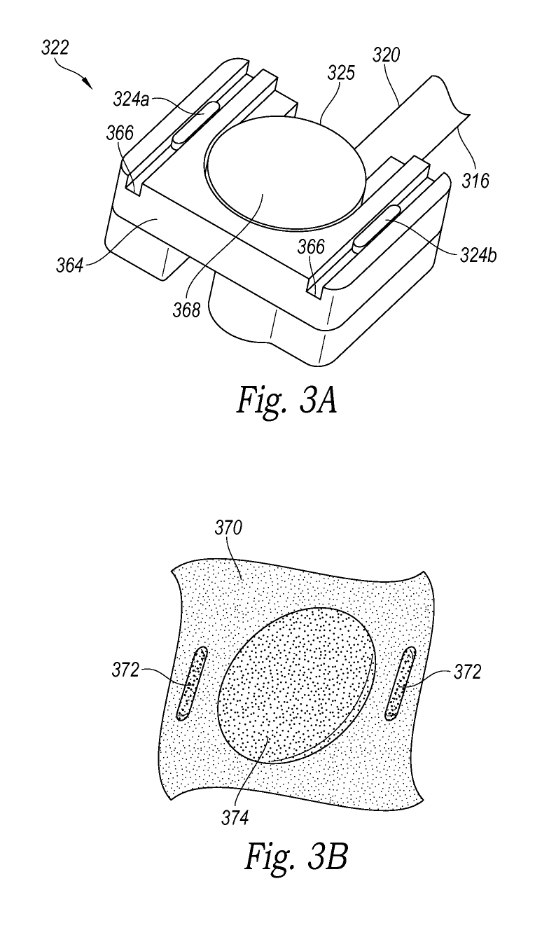

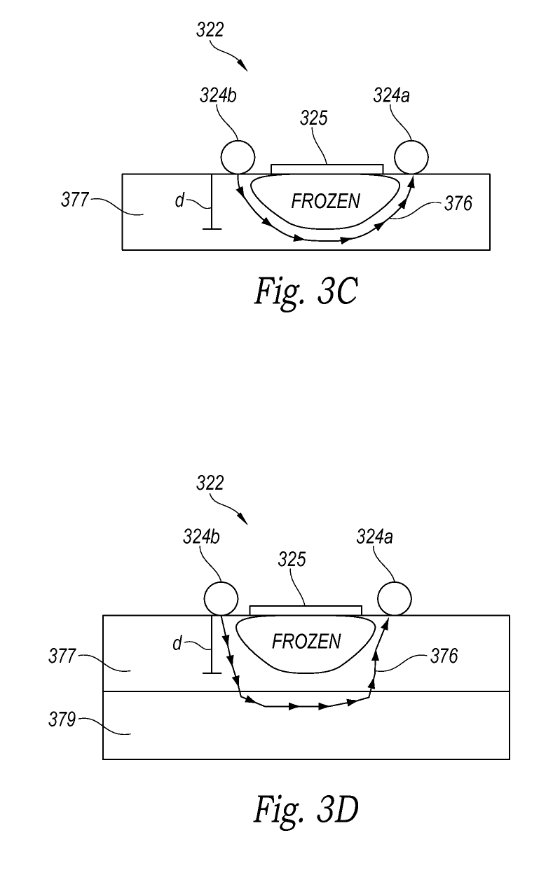

FIG. 3A is an isometric view of a therapeutic assembly 322 configured in accordance with another embodiment of the present technology. The therapeutic assembly 322 can include features generally similar to the features of the therapeutic assembly 122 described above with reference to FIGS. 1A-2C. For example, the therapeutic assembly 322 includes a plurality of energy delivery elements 324 (identified individually as a first energy delivery element 324a and a second energy delivery element 324b) configured to apply hyperthermic energy at a treatment site and a cryogenic applicator 325 configured to providing cooling at least proximate the treatment site. As shown in FIG. 3A, the therapeutic assembly 322 can be at a distal portion 320 of an elongated shaft 316. A proximal portion (not shown) of the elongated shaft 316 can be operably coupled to a handle and/or other features of a treatment device and/or a therapy system (e.g., the treatment device 112 and the therapy system 100 of FIG. 1A) via a proximal portion (not shown) of the shaft 316.

In the embodiment illustrated in FIG. 3A, the cryogenic applicator 325 is a cryoprobe with a substantially rigid and flat applicator surface 368 rather than an expandable member. For example, the cryogenic applicator 325 can include a cryoprobe (e.g., a flat tip cryoprobe, a curved tip cryoprobe, a “J” probe, a pen tip, etc.) and can be operably coupled to a cryoablation cryosurgical system. In other embodiments, the cryogenic applicator 325 can have other suitable configurations depending on the size and location of the treatment site and/or be operably coupled to other suitable cryosurgical systems.

As show in FIG. 3A, the therapeutic assembly 322 can include a housing 364 configured to receive or support the cryogenic applicator 325 such that the applicator surface 368 faces at least generally outward from the housing 364 to deliver cryogenic cooling at a treatment site. The housing 364 can include a plurality of grooves 366 (e.g., channels or slots) configured to retain the energy delivery elements 324 positioned at opposing sides of the cryogenic applicator 325 and facing the same direction as the applicator surface 368. The housing 364 can be made from a variety of materials, such as silicone, polyurethane, polytetrafluoroethylene (PTFE), and/or other suitable materials that are durable enough to withstand the hyperthermic and hypothermic temperatures provided by the therapeutic assembly 122.

In various embodiments, the first and second energy delivery elements 324a and 324b can be configured in corresponding grooves 366 to provide bipolar energy (e.g., RF energy). In other embodiments, both of the energy delivery elements 324a and 324b can be biased at the same polarity, or one of the energy delivery elements 324 can be removed or de-activated and the remaining active energy delivery element 324 is configured to deliver unipolar energy at the treatment site. In such embodiments, a passive return electrode attached exteriorly to the patient can provide a return path for the hyperthermal energy. In still another embodiment, both the energy delivery elements 324 can be biased at a common polarity and the cryogenic applicator 325 can be a third hyperthermal energy delivery element biased at an opposite polarity to provide two bipolar fields.

The therapeutic assembly 322 can be configured to provide cooling at least proximate a treatment site before, during, and/or after the application of therapeutically-effective hyperthermic neuromodulating energy. For example, the cryogenic applicator 325 can be used to pre-treat tissue at the treatment site to at least temporarily modulate or otherwise affect the function of pain neurons, decrease the impedance of the tissue at the treatment site, and/or select a desired target site of the subsequently-formed hyperthermic lesion based on impedance measurements. The cryogenic applicator 325 can also provide cooling during hyperthermic energy delivery to decrease the thermal effects of the ablative hyperthermic energy on tissue proximate the treatment site.

In various embodiments, the therapeutic assembly 322 can be configured to deliver unipolar hyperthermic energy via the first energy delivery element 324a (e.g., operating at about 10 watts). During hyperthermic energy delivery, the cryogenic applicator 325 can cool tissue adjacent the first energy delivery element 324a, which may allow for longer neuromodulation times because of the limited thermal effects to the tissue contacting the first energy delivery element 324a and deeper hyperthermic lesions beyond the cooling zone. To avoid undesirable spikes in impedance caused by low tissue temperatures, the cryogenic applicator 325 can be configured to maintain the temperature at or adjacent the first energy delivery element 324a within a predetermined range. For example, the temperature adjacent the first energy delivery element 324a can be maintained between approximately 15-30° C. When the cryogenic applicator 325 is not being used as a pole of a hyperthermal energy delivery element 324, the cryogenic applicator 325 can be configured to cool the second energy delivery element 324b and/or other suitable structures below freezing (0° C.) such that the energy delivery element 324 or structure can adhere to tissue at the treatment site. The cryogenically cooled second energy delivery element 324b can thereby establish a temporary frozen attachment to the tissue of the treatment site, thereby connecting and stabilizing the therapeutic assembly 322 as a whole to the treatment site, and enhancing the electrical contact between the first energy delivery element 324a and the tissue during hyperthermic energy delivery.

In certain embodiments, the cryogenic applicator 325 can also be used to thermally protect structures (e.g., tissue) proximate the treatment site during hyperthermic energy delivery. Removing heat from tissue to maintain the temperature of the tissue within a predetermined range below body temperature is thought to induce reversible hypothermic effects in tissue (e.g., slowing or temporarily interrupting electrical activity). For example, the cryogenic applicator 325 may be configured to maintain tissue at a temperature between about 5° C. to about 37° C. Upon re-warming, normal cell function of the tissue can be restored. Accordingly, the surface 368 of the cryogenic applicator 325 can be positioned at or on a structure (e.g., a sensitive or vital structure) such that it can be cooled during hyperthermic energy delivery to an adjacent treatment site.

FIG. 3B, for example, is a schematic view of lesions or thermal zones formed on or within tissue 370 at or proximate a treatment site after a neuromodulation and/or ablation procedure using the therapeutic assembly 322 shown in FIG. 3A. As shown in FIG. 3B, the application of RF and/or other hyperthermic energy using the first and second energy delivery elements 324a and 324b (FIG. 3A) can form corresponding hyperthermic lesions 372 or hyperthermic zones that extend a depth into the tissue 370 sufficient to provide therapeutically-effective neuromodulation. The cryogenic applicator 325 (FIG. 3A) can cool a portion of the tissue 370 to a suitable temperature range (e.g., approximately 5-20° C.) during hyperthermic energy delivery to form a protected hypothermic zone 374 that remains at least substantially free from undesirable thermal effects from the hyperthermic energy. The cell function in the cryo-induced hypothermic zone 374 from the cryogenic applicator 325 is expected to return to at least substantially normal cell function upon re-warming because the temperature of the hypothermic zone 374 is selected and maintained (e.g., using temperature sensors) within a range thought to provide only reversible effects. Therefore, the therapeutic assembly 322 can provide therapeutically-effective hyperthermic energy (e.g., RF energy) close to vital and/or sensitive structures with limited effects to the collateral tissue.

In certain embodiments, the therapeutic assembly 322 can be used for RF ablation in cardiac applications, such as for atrioventricular nodal reentrant tachycardias (AVNRTs), atrial fibrillation (AF), and/or ventricular tachycardias (VTs). For example, the cryogenic applicator 325 can be placed on or proximate the adjacent AV node for AVNRT ablation and cooled to a therapeutically-reversible temperature to inhibit ablation of the AV node and electrically protect it from the RF ablation delivered adjacent to the site. In other embodiments, the cryogenic applicator 325 can be used to thermally and electrically protect other structures (e.g., the sinus node, arteries, etc.) adjacent to the site of hyperthermic energy delivery.

FIGS. 3C and 3D are schematic diagrams illustrating a treatment site during neuromodulation (e.g., ablation or non-ablation neuromodulation) using the therapeutic assembly 322 of FIG. 3A (with the housing 364 omitted for clarity) in accordance with an embodiment of the present technology. More specifically, FIG. 3C illustrates the energy delivery elements 324 applying a bipolar energy field (e.g., RF energy) to endocardial tissue 377, to ex vivo tissue, and/or to in vivo tissue when the heat beat has been arrested (e.g., during a cardiopulmonary bypass). FIG. 3D illustrates the energy delivery elements 324 applying a bipolar energy field to epicardial and/or other tissue 377 that can conduct an action potential (e.g., as illustrated by the presence of blood 379 underlying or otherwise surrounding the tissue 377). In other embodiments, the cryo-guided energy delivery method described below can be used with monopolar energy fields at the target tissue.

As shown in FIGS. 3C and 3D, the cryogenic applicator 325 can apply cryogenic cooling to the surface of the tissue 377 until it freezes an underlying and/or adjacent portion of the tissue 377. For example, in certain embodiments, the cryogenic applicator 325 can be cooled to low cryogenic temperatures (e.g., about −55° C. to about −60° C.), and the energy delivery elements 324 can optionally apply a relatively low power (e.g., less than 5 watts) during the cooling to maintain unfrozen tissue sections under the energy delivery elements 324. The cooled portion of the tissue 377 can form a frozen block or ball of tissue extending a depth (d) into the tissue 377. The low-levels of electrical energy applied across the frozen tissue can also be configured to provide real-time impedance measurements that can be used to monitor the development of frozen tissue. Once the cryogenically cooled portion of the tissue 377 reaches a predetermined temperature (e.g., about −50° C.) or the frozen portion reaches a predetermined depth (d), the power to the energy delivery elements 324 can be increased to provide therapeutically-effective ablation. Optionally, the power may be manually or automatically adjusted to maintain the electrode temperatures below a threshold (e.g., below about 70° C.) to inhibit the hyperthermic energy application from melting the adjacent frozen tissue mass.

The frozen tissue has a relatively high impedance that can direct or otherwise manipulate the application of hyperthermic energy. As shown in FIGS. 3C and 3D, for example, when the energy delivery elements 324 apply a current 376 across the tissue 377, the hyperthermic energy flows along the path of least resistance (least impedance) around the frozen tissue to at least the depth (d) into the tissue 377. As shown in FIG. 3D, when the tissue 377 is proximate a volume of blood 379, the conductivity of the blood 379 can draw the bipolar current 376 deeper into the tissue 377 to increase the hyperthermic lesion depth. The cryogenic applicator 325 can thus be used to freeze or otherwise sufficiently cool the tissue and direct the application of energy, e.g., to a desired depth within the tissue 377.

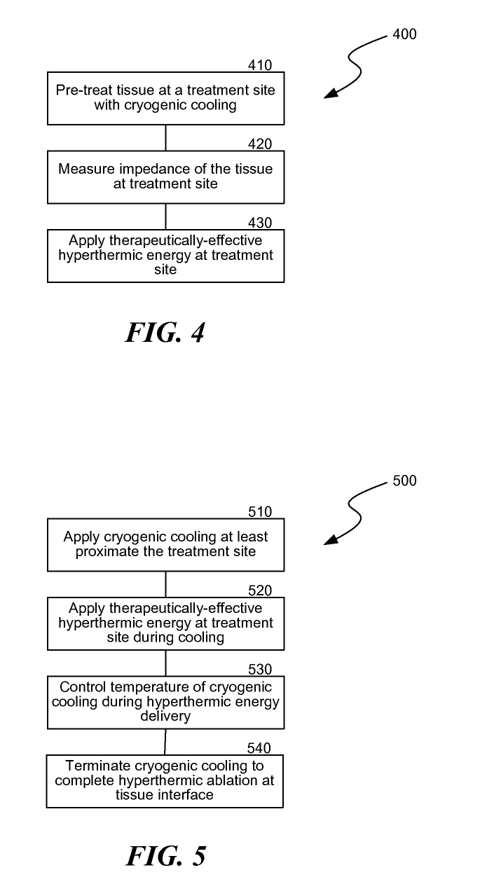

FIGS. 4-6 are block diagrams illustrating therapeutic methods that use both hyperthermic energy and cryogenic cooling in accordance with embodiments of the present technology. The methods can be performed by the therapeutic assemblies 122 and 322 described above with reference to FIGS. 2A-3D, combinations thereof, and/or other suitable hyperthermic and/or hypothermic devices.

FIG. 4 is a block diagram illustrating a therapeutic method 400 that uses a cryogenic cooling phase to pre-treat tissue at a treatment site (block 410) before applying hyperthermic energy in accordance with an embodiment of the present technology. The cryogenic cooling can be provided by an expandable cryogenic balloon applicator (e.g., similar to the cryogenic applicator 125 of FIGS. 2A-2C), a cryoprobe applicator (e.g., similar to the cryogenic applicator 325 of FIG. 3A), and/or other suitable cryogenic cooling devices. Cooling can be applied at a predetermined temperature (e.g., about −60° C. to about 0° C.) and for a predetermined time interval (e.g., 1 minute, 2 minutes, 4 minutes, 8 minutes, etc.), and in some embodiments cooling periods may be interposed with thawing periods (e.g., 1 minute cool, thaw to body temperature, 1 minute cool, etc.). The cryogenic pre-treatment of the tissue can modulate or otherwise alter pain neurons at the treatment site and inhibit their communication with the CNS to reduce the level of pain experienced by the patient during subsequent applications of hyperthermic energy. For example, the method 400 can be used during renal neuromodulation procedures to pre-treat the tissue of a renal artery or an ostium of a renal artery because efferent pain sensors and fibers are located close to the targeted sympathetic nerves. In addition, cryogenically pre-treating the tissue can alter the cell walls of the tissue, and thereby decrease the electrical impedance of the tissue and thereby beneficially direct the path of hyperthermic energy.

After cryogenic pre-treatment, the method 400 can optionally include measuring the tissue impedance at the treatment site to determine the effects of the pre-treatment (block 420). Impedance measurements can be taken by applying a signal across the pre-treated cryo-cooled area using electrodes and/or other suitable impedance measurement devices. The measured impedance can be used to determine whether the impedance of the tissue has been reduced to a desired extent and/or to plan treatment protocols for subsequent hyperthermic ablation (e.g., estimating ablation times, etc.).

The method 400 can further include applying therapeutically-effective hyperthermic energy at the treatment site (block 430). The hyperthermic energy can be in the form of a monopolar RF energy field, a bipolar RF energy field, microwave energy, direct current (DC), ultrasound energy, high intensity focused (HIFU) energy, optical energy, hyperthermic chemical energy, and/or other suitable forms of hyperthermic energy that can provide therapeutically-effective neuromodulation. The hyperthermic energy can be delivered via various suitable hyperthermic energy delivery devices, such as RF electrodes, an intravascular ablation device, and/or a monopolar ablation pen (e.g., a CARDIOBLATE ablation pen available from Medtronic, Inc. of Minneapolis, Minn.). The power at which the hyperthermic energy is applied, the length of the time interval for energy delivery, and/or other energy delivery parameters can be selected based on the characteristics of the treatment site and the desired therapeutic effect (e.g., lesion depth). For example, in some embodiments hyperthermic energy (e.g., RF energy) can be applied at a power of about 10-20 watts for time intervals lasting about 20-60 seconds, or in other embodiments at less than 10 watts for more than 60 seconds. In other embodiments, the method 400 can include other suitable hyperthermic ablation times and/or power levels. In the method 400, the power level and/or the duration of hyperthermic energy delivery can be decreased in comparison to conventional ablation methods because the pre-treated cryogenically-cooled tissue provides enhanced transmission of the hyperthermic energy. Therefore, the method 400 can provide for efficient delivery of hyperthermic energy to the treatment site, and can do so with less pain experienced by the patient.

FIG. 5 is a block diagram illustrating a therapeutic method 500 that uses concurrent delivery of hyperthermic energy and cryogenic cooling in accordance with an embodiment of the present technology. The method 500 can include applying cryogenic cooling at least proximate a treatment site (block 510), and applying therapeutically-effective hyperthermic energy to the treatment site during at least a portion of the time while applying cooling (block 520). The method 500 can use generally similar devices and modalities to provide cryogenic cooling and hyperthermic energy as the devices and modalities described with reference to the method 400 of FIG. 4. The method 500 of FIG. 5 applies cryogenic and hyperthermic energy during at least partially overlapping time intervals, or in some embodiments the cryogenic and hyperthermic energies can be applied simultaneously. For example, cryogenic cooling can be delivered to tissue at least proximate the treatment site to maintain relatively low temperatures at the surface of the tissue, and thereby inhibit the hyperthermic energy delivery (e.g., occurring simultaneously) from causing undesirable thermal effects to the tissue. Cryogenic cooling can also be applied to the device delivering the hyperthermic energy and/or portions thereof. For example, cryogenic cooling can be applied to an energy delivery element (e.g., a monopolar RF electrode) to reduce the operating temperature of the energy delivery element to inhibit hyperthermic effects to the adjacent tissue. By controlling the thermal effects to the surface of the tissue, the method 500 can apply hyperthermic energy for longer periods of time, and therefore reach greater lesion depths for neuromodulation.

Despite the benefits of cryogenic cooling during hyperthermic energy delivery, too much cooling can increase the impedance of the tissue and inhibit energy delivery. Accordingly, the method 500 can also include controlling the temperature of the cryogenic cooling during hyperthermic energy delivery (block 530). For example, the temperature proximate the hyperthermic energy delivery element(s) can be kept at low, but relatively moderate temperature levels (e.g., above freezing; about 15-30° C.) to prevent an increase of the tissue impedance proximate energy delivery. The temperature can be adjusted manually or automatically using a controller operably coupled the cryogenic cooling device and a temperature sensor proximate the treatment site. In embodiments including structures (e.g., non-active electrodes) that are cooled to adhere to tissue for enhanced electrical contact, the step in block 530 can also or alternatively include maintaining the temperature of the structure below the freezing temperature of the tissue.

As further shown in FIG. 5, the method 500 can also optionally include terminating cryogenic cooling before terminating hyperthermic energy delivery (block 540). This allows the tissue immediately adjacent the energy delivery element to warm quickly and thereby complete the hyperthermic ablation at the tissue interface. For example, in certain embodiments, the cryogenically cooled energy delivery element may not reach ablative temperatures at the interface with the tissue, and therefore the cryogenic cooling can be suspended to complete the hyperthermic lesion.

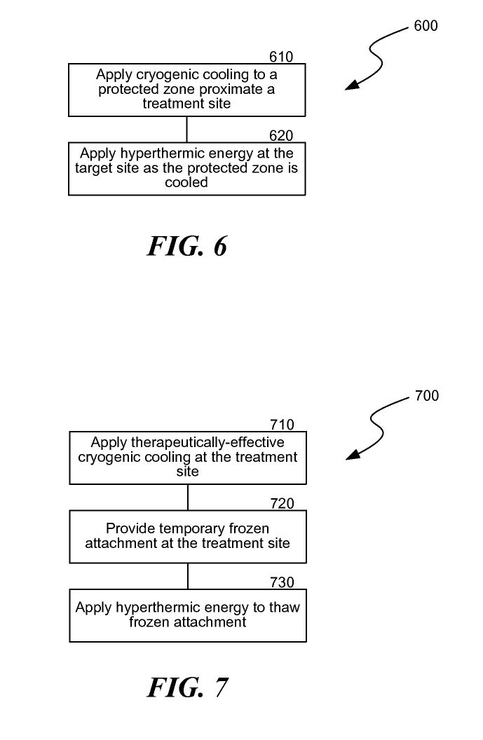

FIG. 6 is a block diagram illustrating a therapeutic method 600 that uses concurrent delivery of hypothermic energy and cryogenic cooling in accordance with another embodiment of the present technology. The method 600 can include cryogenically cooling tissue at a protected zone or area proximate a treatment site (block 610), and applying hyperthermic energy at the treatment site as the protected zone is cooled (block 620). The protected zone can be cooled to a temperature (e.g., about 5-37° C.) that causes reversible hypothermic effects in the tissue such that function of the tissue can return, at least partially, upon reheating. Accordingly, the method 600 uses cryogenic cooling to protect tissue and/or structures adjacent to the treatment site from undesirable thermal effects and/or electrical interference caused by the delivery of hyperthermic energy to the treatment site.

FIG. 7 is a block diagram illustrating a therapeutic method 700 that uses cryogenic cooling and hyperthermic energy in accordance with yet another embodiment of the present technology. The method 700 can include applying therapeutically-effective cryogenic cooling to target tissue to modulate nerves at a treatment site (block 710). Therapeutically-effective cryogenic cooling can be provided by a cryogenic applicator, such as a cryoballoon or a cryoprobe. The cryogenic applicator may be configured to form a temporary frozen attachment with adjacent tissue (block 720), and thereby provide at least substantially stable contact with the tissue to facilitate the cryogenic cooling. After neuromodulation via the cryogenic cooling, hyperthermic energy (e.g., RF energy) can be applied proximate the frozen attachment between the tissue and the cryogenic applicator to quickly thaw the ice bond (block 730), and allow the cryogenic applicator to be moved away from the treatment site. The hyperthermic energy can be less than that required to modulate renal nerves and may be provided by one or more RF electrodes and/or other suitable heating components. The method 700 is expected to substantially reduce the overall cryoablation time, as it often takes a substantial portion of the procedure time to thaw the ice bond (e.g., the same amount of time it takes to provide cryotherapeutic neuromodulation). In other embodiments, cryogenic cooling can be used to provide a temporary frozen attachment to non-target tissue proximate the treatment site (i.e., not to provide neuromodulation) while therapeutically-effective hyperthermic energy is applied to stabilize the treatment device at the target site. After the desired therapeutic effect is provided (e.g., renal denervation), the cryogenic cooling can cease and the hyperthermic energy can continue to be applied (e.g., at a lower power) to thaw the frozen bond.

### IV. RELATED ANATOMY AND PHYSIOLOGY

The Sympathetic Nervous System (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up- and down-regulating many homeostatic mechanisms in living organisms. Fibers from the SNS innervate tissues in almost every organ system, providing at least some regulatory function to physiological features as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

1. The Sympathetic Chain

As shown in FIG. 8, the SNS provides a network of nerves that allows the brain to communicate with the body. Sympathetic nerves originate inside the vertebral column, toward the middle of the spinal cord in the intermediolateral cell column (or lateral horn), beginning at the first thoracic segment of the spinal cord and are thought to extend to the second or third lumbar segments. Because its cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they quickly separate out through white rami connectors which connect to either the paravertebral (which lie near the vertebral column) or prevertebral (which lie near the aortic bifurcation) ganglia extending alongside the spinal column.

In order to reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other components of the peripheral nervous system, these synapses are made at sites called ganglia, discussed above. The cell that sends its fiber is called a preganglionic cell, while the cell whose fiber leaves the ganglion is called a postganglionic cell. As mentioned previously, the preganglionic cells of the SNS are located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle and inferior), which sends sympathetic nerve fibers to the head and thorax organs, and the celiac and mesenteric ganglia (which send sympathetic fibers to the gut).

2. Innervation of the Kidneys

As FIG. 8 shows, the kidney is innervated by the renal plexus (RP), which is intimately associated with the renal artery. The renal plexus (RP) is an autonomic plexus that surrounds the renal artery and is embedded within the adventitia of the renal artery. The renal plexus (RP) extends along the renal artery until it arrives at the substance of the kidney. Fibers contributing to the renal plexus (RP) arise from the celiac ganglion, the superior mesenteric ganglion, the aorticorenal ganglion and the aortic plexus. The renal plexus (RP), also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, first lumbar splanchnic nerve, second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

3. Renal Sympathetic Neural Activity

Messages travel through the SNS in a bidirectional flow. Efferent messages may trigger changes in different parts of the body simultaneously. For example, the sympathetic nervous system may accelerate heart rate; widen bronchial passages; decrease motility (movement) of the large intestine; constrict blood vessels; increase peristalsis in the esophagus; cause pupil dilation, piloerection (goose bumps) and perspiration (sweating); and raise blood pressure. Afferent messages carry signals from various organs and sensory receptors in the body to other organs and, particularly, the brain.

Hypertension, heart failure and chronic kidney disease are a few of many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure), and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end stage renal disease are characterized by heightened sympathetic nervous activation. In patients with end stage renal disease, plasma levels of norepinephrine above the median have been demonstrated to be predictive for both all-cause death and death from cardiovascular disease. This is also true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well known adverse consequences of chronic sympathetic over activity, such as hypertension, left ventricular hypertrophy, ventricular arrhythmias, sudden cardiac death, insulin resistance, diabetes, and metabolic syndrome.

(i) Renal Sympathetic Efferent Activity

Sympathetic nerves to the kidneys terminate in the blood vessels, the juxtaglomerular apparatus and the renal tubules. Stimulation of the renal sympathetic nerves causes increased renin release, increased sodium (Na+) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation is likely a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release) and diuretics (intended to counter the renal sympathetic mediated sodium and water retention). However, the current pharmacologic strategies have significant limitations including limited efficacy, compliance issues, side effects and others.

(ii) Renal Sensory Afferent Nerve Activity

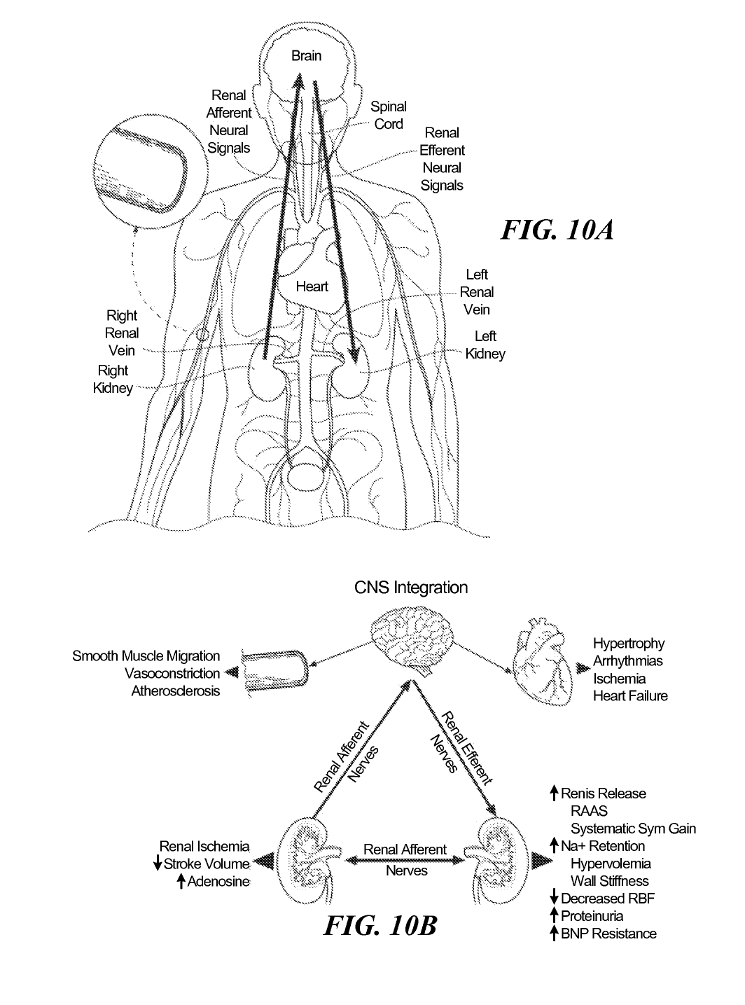

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication. As shown in FIGS. 10A and 10B, this afferent communication might be from the kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, volume retention and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

B. Additional Clinical Benefits of Renal Denervation

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end stage renal disease, inappropriate fluid retention in heart failure, cardio-renal syndrome, and sudden death. Since the reduction of afferent neural signals contributes to the systemic reduction of sympathetic tone/drive, renal denervation might also be useful in treating other conditions associated with systemic sympathetic hyperactivity. Accordingly, renal denervation may also benefit other organs and bodily structures innervated by sympathetic nerves, including those identified in FIG. 8. For example, as previously discussed, a reduction in central sympathetic drive may reduce the insulin resistance that afflicts people with metabolic syndrome and Type II diabetics. Additionally, patients with osteoporosis are also sympathetically activated and might also benefit from the down regulation of sympathetic drive that accompanies renal denervation.

C. Achieving Intravascular Access to the Renal Artery

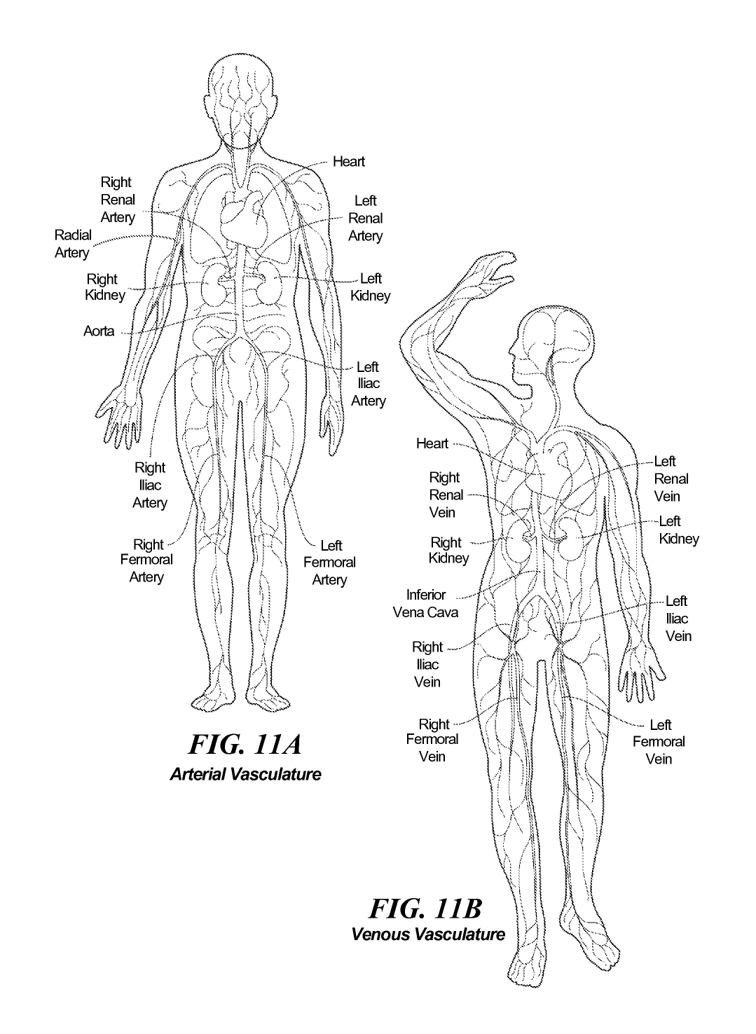

In accordance with the present technology, neuromodulation of a left and/or right renal plexus (RP), which is intimately associated with a left and/or right renal artery, may be achieved through intravascular access. As FIG. 11A shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

As FIG. 11B shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

As will be described in greater detail later, the femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters introduced via these access points may be passed through the subclavian artery on the left side (or via the subclavian and brachiocephalic arteries on the right side), through the aortic arch, down the descending aorta and into the renal arteries using standard angiographic technique.

D. Properties and Characteristics of the Renal Vasculature

Since neuromodulation of a left and/or right renal plexus (RP) may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, etc. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Apparatus, systems and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. For example, navigation can be impeded by the tight space within a renal artery, as well as tortuosity of the artery. Furthermore, establishing consistent contact is complicated by patient movement, respiration, and/or the cardiac cycle because these factors may cause significant movement of the renal artery relative to the aorta, and the cardiac cycle may transiently distend the renal artery (i.e. cause the wall of the artery to pulse).

Even after accessing a renal artery and facilitating stable contact between neuromodulatory apparatus and a luminal surface of the artery, nerves in and around the adventia of the artery should be safely modulated via the neuromodulatory apparatus. Effectively applying thermal treatment from within a renal artery is non-trivial given the potential clinical complications associated with such treatment. For example, the intima and media of the renal artery are highly vulnerable to thermal injury. As discussed in greater detail below, the intima-media thickness separating the vessel lumen from its adventitia means that target renal nerves may be multiple millimeters distant from the luminal surface of the artery. Sufficient energy should be delivered to or heat removed from the target renal nerves to modulate the target renal nerves without excessively cooling or heating the vessel wall to the extent that the wall is frozen, desiccated, or otherwise potentially affected to an undesirable extent. A potential clinical complication associated with excessive heating is thrombus formation from coagulating blood flowing through the artery. Given that this thrombus may cause a kidney infarct, thereby causing irreversible damage to the kidney, thermal treatment from within the renal artery should be applied carefully. Accordingly, the complex fluid mechanics and thermodynamic conditions present in the renal artery during treatment, particularly those that may impact heat transfer dynamics at the treatment site, may be important in applying energy (e.g., heating thermal energy) and/or removing heat from the tissue (e.g., cooling thermal conditions) from within the renal artery.

The neuromodulatory apparatus should also be configured to allow for adjustable positioning and repositioning of the energy delivery element within the renal artery since location of treatment may also impact clinical efficacy. For example, it may be tempting to apply a full circumferential treatment from within the renal artery given that the renal nerves may be spaced circumferentially around a renal artery. In some situations, a full-circle lesion likely resulting from a continuous circumferential treatment may be potentially related to renal artery stenosis. Therefore, the formation of more complex lesions along a longitudinal dimension of the renal artery and/or repositioning of the neuromodulatory apparatus to multiple treatment locations may be desirable. It should be noted, however, that a benefit of creating a circumferential ablation may outweigh the potential of renal artery stenosis or the risk may be mitigated with certain embodiments or in certain patients and creating a circumferential ablation could be a goal. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging. Manipulation of a device in a renal artery should also consider mechanical injury imposed by the device on the renal artery. Motion of a device in an artery, for example by inserting, manipulating, negotiating bends and so forth, may contribute to dissection, perforation, denuding intima, or disrupting the interior elastic lamina.

Blood flow through a renal artery may be temporarily occluded for a short time with minimal or no complications. However, occlusion for a significant amount of time should be avoided because to prevent injury to the kidney such as ischemia. It could be beneficial to avoid occlusion all together or, if occlusion is beneficial to the embodiment, to limit the duration of occlusion, for example to 2-5 minutes.

Based on the above described challenges of (1) renal artery intervention, (2) consistent and stable placement of the treatment element against the vessel wall, (3) effective application of treatment across the vessel wall, (4) positioning and potentially repositioning the treatment apparatus to allow for multiple treatment locations, and (5) avoiding or limiting duration of blood flow occlusion, various independent and dependent properties of the renal vasculature that may be of interest include, for example, (a) vessel diameter, vessel length, intima-media thickness, coefficient of friction, and tortuosity; (b) distensibility, stiffness and modulus of elasticity of the vessel wall; (c) peak systolic, end-diastolic blood flow velocity, as well as the mean systolic-diastolic peak blood flow velocity, and mean/max volumetric blood flow rate; (d) specific heat capacity of blood and/or of the vessel wall, thermal conductivity of blood and/or of the vessel wall, and/or thermal convectivity of blood flow past a vessel wall treatment site and/or radiative heat transfer; (e) renal artery motion relative to the aorta induced by respiration, patient movement, and/or blood flow pulsatility: and (f) as the take-off angle of a renal artery relative to the aorta. These properties will be discussed in greater detail with respect to the renal arteries. However, dependent on the apparatus, systems and methods utilized to achieve renal neuromodulation, such properties of the renal arteries, also may guide and/or constrain design characteristics.

As noted above, an apparatus positioned within a renal artery should conform to the geometry of the artery. Renal artery vessel diameter, D_{RA}, typically is in a range of about 2-10 mm, with most of the patient population having a D_{RA} of about 4 mm to about 8 mm and an average of about 6 mm. Renal artery vessel length, L_{RA}, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >5 mm from inner wall of the renal artery) to avoid non-target tissue and anatomical structures such as the renal vein.

An additional property of the renal artery that may be of interest is the degree of renal motion relative to the aorta induced by respiration and/or blood flow pulsatility. A patient's kidney, which is located at the distal end of the renal artery, may move as much as 4″ cranially with respiratory excursion. This may impart significant motion to the renal artery connecting the aorta and the kidney, thereby requiring from the neuromodulatory apparatus a unique balance of stiffness and flexibility to maintain contact between the energy delivery element and the vessel wall during cycles of respiration. Furthermore, the take-off angle between the renal artery and the aorta may vary significantly between patients, and also may vary dynamically within a patient, e.g., due to kidney motion. The take-off angle generally may be in a range of about 30°-135°.

### VI. CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A treatment device, comprising:
a shaft including a proximal portion and a distal portion, wherein the shaft is configured to insert the distal portion at a treatment site at least proximate a renal artery;
a therapeutic assembly extending from the distal portion of the shaft, the therapeutic assembly comprising—
an energy delivery element configured to apply therapeutically-effective hyperthermic energy to tissue at the treatment site to modulate renal nerves proximate the treatment site; and
a cryogenic applicator configured to cool tissue at least proximate the treatment site.

2. The treatment device of claim 1 wherein:
the therapeutic assembly has a delivery state and a deployed state;
the energy delivery element comprises a plurality of energy delivery elements including at least a first electrode and a second electrode, wherein the first and second electrodes are configured to deliver therapeutically-effective radiofrequency energy to the treatment site when the therapeutic assembly is in the deployed state;
the cryogenic applicator includes an expandable member having an outer surface, the first and second electrodes being arranged about the outer surface, wherein the expandable member is configured to cool a zone at least proximate the treatment site when the therapeutic assembly is in the deployed state; and
the treatment device further comprises—
a supply lumen extending along at least a portion of the shaft, the supply lumen being configured to receive a refrigerant in an at least substantially liquid state, and
an exhaust lumen extending along at least a portion of the shaft and in fluid communication with the expandable member.

3. The treatment device of claim 1 wherein:
the energy delivery element comprises a plurality of energy delivery elements including at least a first electrode and a second electrode, wherein the first and second electrodes are configured to apply therapeutically-effective radiofrequency energy to the treatment site; and
the cryogenic applicator comprises a cryoprobe having a surface configured to cool a zone at least proximate the treatment site, wherein the first and second electrodes are spaced laterally apart from one another at opposing sides of the surface.

4. The treatment device of claim 1 wherein:
the cryogenic applicator comprises a cryoprobe;
the energy delivery element comprises a plurality of electrodes configured to deliver therapeutically-effective radiofrequency energy to the treatment site; and
the plurality of electrodes include at least a first electrode and a second electrode spaced laterally apart from one another with the cryogenic applicator between the first and second electrodes.

5. The treatment device of claim 1, further comprising a controller operably coupled to the cryogenic applicator and the energy delivery element, wherein the controller is configured to cool the treatment site via the cryogenic applicator before applying hyperthermic energy to the treatment site.

6. The treatment device of claim 5 wherein the cryogenic applicator is configured to cool the treatment site to decrease an impedance of the tissue at the treatment site.

7. The treatment device of claim 5 wherein the cryogenic applicator is configured to cool the treatment site to modulate pain neurons at the treatment site.

8. The treatment device of claim 1 wherein:
the therapeutic assembly has a delivery state and a deployed state; and
the energy delivery element comprises a plurality of electrodes arranged in a helical pattern when the therapeutic assembly is in the deployed state.

9. A treatment device, comprising:
a shaft including a proximal portion and a distal portion, wherein the shaft is configured to insert the distal portion at a treatment site at least proximate a renal artery;
an energy delivery element attached to the distal portion of the shaft and configured to deliver therapeutically-effective energy that modulates nerves that innervate the kidney, wherein the energy delivery element is configured to be operably coupled to a hyperthermic energy source positioned at the proximal portion of the shaft; and
a cryogenic applicator attached to the distal portion of the shaft and in fluid communication with a refrigerant source positioned at the proximal portion of the shaft.

10. The treatment device of claim 9 wherein the shaft, the energy delivery element, and the cryogenic applicator are sized to fit slideably within a guide catheter having a lumen diameter of approximately 1.80 mm (0.071 inch).

11. The treatment device of claim 9 wherein the expandable member is configured to at least substantially occlude the renal artery in the deployed state.

12. The treatment device of claim 9 wherein the cryogenic applicator comprises a cryoprobe having an applicator surface configured to cool a zone, and wherein the energy delivery element is spaced laterally apart from the zone.

13. The treatment device of claim 9 wherein the cryogenic applicator is configured to cool the energy delivery element during delivery of hyperthermic energy.

14. The treatment device of claim 9 wherein the cryogenic applicator is configured to cool the treatment site before delivery of hyperthermic energy.

15. A method of treating a patient, comprising:
inserting a therapeutic assembly to a treatment site in a renal artery, wherein the therapeutic assembly extends from a distal portion of an elongated shaft;
cooling a zone at least proximate the treatment site using a cryogenic applicator of the therapeutic assembly; and
applying therapeutically-effective hyperthermic energy to the treatment site to cause renal nerve modulation using an energy delivery element of the therapeutic assembly.

16. The method of claim 15 wherein cooling the zone at least proximate the treatment site comprises cooling tissue at least proximate the zone for a time interval before applying the therapeutically-effective hyperthermic energy to the treatment site.

17. The method of claim 15 wherein cooling the zone at least proximate the treatment site comprises cooling tissue at the zone for at least 1 minute at a temperature of less than 0° C. before applying the therapeutically-effective hyperthermic energy to the renal nerves.

18. The method of claim 15 wherein cooling the zone comprises:
cooling the zone for a first time interval having a duration of at least 1 minute;
thawing the zone for a second time interval having a duration of at least 1 minute; and
cooling the zone of tissue for a second time interval having a duration of at least 1 minute, wherein cooling and thawing occur before applying the therapeutically-effective hyperthermic energy at the treatment site.

19. The method of claim 15 wherein the energy delivery element is a first energy delivery element, and wherein cooling the zone comprises:
freezing a portion of tissue in the zone to the cryogenic applicator before applying the therapeutically-effective hyperthermic energy at the treatment site; and
applying a low-level of energy across the portion of tissue using the first energy delivery element and a second energy delivery element, the first energy delivery element being spaced laterally apart from the second energy delivery element by the portion of tissue, wherein the application of low-level energy is configured to prevent freezing the tissue at the first and second energy delivery elements.

20. The method of claim 15 wherein cooling the zone comprises freezing pain neurons at least proximate the target site, and wherein cooling occurs before the application of hyperthermic energy.

21. The method of claim 15 wherein cooling the zone at least proximate the treatment site comprises decreasing the impedance characteristics of the tissue at the treatment site, wherein cooling occurs before the application of hyperthermic energy.

22. The method of claim 15 wherein cooling the zone at least proximate the treatment site comprises:
positioning the cryogenic applicator on a protected portion of tissue proximate the treatment site; and
cooling the protected portion of tissue during the application of hyperthermic energy.

23. The method of claim 22 wherein cooling the protected portion comprises maintaining a tissue temperature between about 5° C. and about 35° C. at the protected portion during the application of hyperthermic energy.

24. The method of claim 25 wherein cooling the zone at least proximate the treatment site comprises:
cooling the treatment site using the cryogenic applicator before applying hyperthermic energy;
measuring impedance at the treatment site resulting from the cooling; and
applying additional cooling to the treatment site using the cryogenic applicator when the treatment site has a measured impedance above a predetermined threshold, wherein the cooling and measuring steps are repeated until an impedance is measured below the predetermined threshold.

25. The method of claim 15 wherein:
locating the therapeutic assembly at the treatment site comprises locating a first energy delivery element at a first portion of the treatment site, and locating a second energy delivery element at a second portion of the treatment site spaced apart from the first portion;
cooling the zone comprises cooling the first energy delivery element to approximately 5-35° C. during the application of hyperthermic energy by the first energy delivery element, and cooling the second energy delivery element to below 0° C.; and
applying therapeutically-effective hyperthermic energy at the treatment site comprises applying hyperthermic energy at the treatment site using only the first energy delivery element.

26. The method of claim 15 wherein:
cooling the zone comprises freezing a portion of the cryogenic applicator to tissue at least proximate the treatment site to form a temporary bond between the therapeutic assembly and the cryogenic applicator; and
the method further comprises applying hyperthermic energy to the temporary bond after renal nerve modulation to thaw the bond.

27. A method of treating a patient, comprising:
locating a therapeutic assembly at a treatment site at least proximate a renal artery, wherein the therapeutic assembly is at a distal portion of an elongated shaft;
applying therapeutically-effective cryogenic cooling at the treatment site using a cryogenic applicator of the therapeutic assembly to modulate nerves that innervate the kidney;
freezing at least a portion of the cryogenic applicator to tissue at the treatment site to form a temporary frozen attachment; and
applying hyperthermic energy at least proximate the temporary frozen attachment using an energy delivery element of the therapeutic assembly to thaw the frozen attachment.
